(19) United States
(12) Patent Application Publication
(10) Pub. No.: US 2006/0105464 A1
(43) Pub. Date: May 18, 2006

(54) Quantitative determination of analyte

(76) Inventors: Jorg Friedrich Wilhelm Weber, Baton Rouge, LA (US); John T. Rizzo, Baton Rouge, LA (US); James T. Ritchie, Zachary, LA (US); Alan B. Berry, Denham Springs, LA (US); Allen D. Godwin, Seabrook, TX (US)

Correspondence Address: ExxonMobil Chemical Company;Law Technology, P.O. Box 2149, Baytown, TX, 77522-2149, US

(21) Appl. No.: 10/988,069

(22) Filed: Nov. 12, 2004

(51) Int. Cl. G01N 33/00

(52) U.S. Cl. 436/128

(57) ABSTRACT

The invention relates to an analytical technique for the quantitative determination of an analyte and a reagent solution useful in said quantitative determination. The analytical technique is conveniently adapted for quantitative determination of carbonyl and even more particularly adapted for finishing an alcohol produced in the Oxo Process.

### FIELD OF THE INVENTION

The invention relates to an analytical technique for the quantitative determination of an analyte and a reagent solution useful in said quantitative determination.

### BACKGROUND OF THE INVENTION

An important route to C₃ and higher alcohols involves hydroformylation of alpha-olefins, such as ethylene, propylene, and butene-1, to yield the corresponding aldehyde having one more carbon atom than the starting olefin, followed by hydrogenation to the alcohol. The commercially important Oxo Process produces such alcohols, which find uses in plastics, soaps, lubricants, and other products. Thus, hydroformylation of ethylene yields propionaldehyde and propylene yields a mixture of n- and iso-butyraldehyde (with the n-isomer usually predominating), followed by catalytic hydrogenation to the corresponding alcohols, e.g. n-propanol and n-butanol. Synthetic alcohols, particularly those in the range of about 8 to 13 carbon atoms (C8-C13), are used as plasticizers for poly(vinyl chloride) and the like. By way of example, the important plasticizer alcohol, 2-ethylhexanol, is made by alkali-catalyzed condensation of n-butyraldehyde to yield the unsaturated aldehyde, 2-ethyl-hex-2-enal, which is then hydrogenated to yield the desired 2-ethylhexanol.

Historically the preferred catalysts for such aldehyde hydrogenation reactions are the Group VIII metal catalysts, such as cobalt, nickel, palladium, platinum, or rhodium. Numerous other systems have been proposed, with varying degrees of success. The Oxo Process and variations thereon are the subject of numerous patents and patent applications, recent examples of which are WO 03/082788 and 03/082789.

Synthetic alcohols are typically plagued with the problem of undesirable color and color forming impurities, e.g., aldehydes and ketones. Many methods have been tried to mitigate the problem, for example, treatment with reducing agents, such as hydrogen in the presence of a catalyst such as zinc and copper catalyst, Raney nickel catalyst, zirconium promoted nickel-kieselguhr catalyst, or the like, treatment with borohydrides such as sodium borohydride, and also ozone treatments. See, for instance, U.S. Pat. No. 3,642,915.

As an example of a commercial process, the crude alcohol product from the hydrogenation section of the Oxo Process, containing color and color-forming impurities, is passed through a finishing section, where it is treated with sodium borohydride. The reactivity of sodium borohydride towards aldehydes and ketones (if present) is much greater than the reactivity of sodium borohydride with the active hydrogen of the alcohol or the ester carbonyl. Sodium borohydride reduces aldehydes and ketones to the corresponding alcohols. Excess sodium borohydride will lead to the formation of particulates in the product alcohol. It can also slow down the reaction to form plasticizers in the next production step. It may also lead to a decrease in resistivity in products used for wire and cable insulation. In the case where hydrogen is used in the finishing section, excess use of hydrogen is disadvantageous at least because of the expense.

The amount of reducing agent to use in the finishing section will depend on the amount of aldehydes and ketones in the crude product. Accordingly, analysis of the crude product for carbonyl content is important to avoid over- or under-treatment in the finishing section.

The amount of residual aldehyde and ketone may be expressed as a carbonyl number. The theoretical carbonyl number (TCBN) of a material is traditionally reported in mg KOH per gram of sample. This originated from the fact that historically KOH was used to titrate the HCL liberated when the carbonyl compound reacted with hydroxylamine hydrochloride. The theoretical value for a pure carbonyl compound is expressed by the following formula:
TCBN=(FW_{KOH}/FW_{CARBONYL COMPOUND})×(N_{CARBONYL COMPOUND})×1000 mg/g
where FW is the formula weight of the species specified in the equation. N is the number of active carbonyl groups in the carbonyl compound. The TCBN for pure 2-octanone, typically used as a calibration standard, is 438. The carbonyl number (CBN) for a standard is expressed by the following formula:
CBN═(W×TCBN×P)/T
where W the weight of carbonyl compound; P is the percent purity of carbonyl compound; T is the total weight of standard. The CBN for 98% pure 2-octanone is 429 (W/T=1). The units for both TCBN and CBN is mg KOH/g, which are typically omitted in reporting the respective numbers.

The CBN value for an unknown sample maybe obtained via direct titration, by way of example, with hydroxylammoniumchloride to form an oxime and free hydrochloric acid followed by pontentiometric titration of the free hydrochloric acid with an alcoholic solution of tetra-n-butyl ammonium hydroxide (c.f. ISO 1843). or an inferential technique using, by way of example, an extractive method followed by spectrophotometric determination as set forth, for instance, by Lohman, Spectroscopic Determination of Carbonyl Oxygen, Analytical Chemistry, Vol. 30, No. 5, May 1958, p. 972-974, or a non-extractive technique using spectrophotometric determination as set forth, for instance, by Bartkiewicz and Kenyon, in Anal. Chem. Vol. 35, No. 3, March 1963. These methods are laborious and at best the results are obtained on the order of one hour after the sample is taken. While the analysis is going on, the commercial process continues with possible wasteful use of treating chemicals and/or poor quality control of the product alcohol, as previously discussed.

As an example, a current analytical technique, described in the document BRCP 4589, available from ExxonMobil Chemical Company, Baton Rouge, La., uses a Bran and Luebbe AA2 or AA3 equipped with a colorimeter. The reagent solution is prepared as follows: 40 mL conc. HCl is added slowly to 3800 mL denatured alcohol solvent, followed by 4 grams 2,4-dinitrophenylhydrazine (DNPH), which reacts specifically with aldehydes and ketones. 200 mL Water is added. Carboxylic acids and esters are unreactive towards DNPH and do not contribute to the carbonyl number. The sample to be tested is then added to the reagent (after first filtering to remove suspended matter, if any). The resulting hydrazone derivative is then treated with base (e.g., KOH) to immediately form a dark-colored entity. The dark color slowly turns into a yellow-brownish color. The colorimeter, properly calibrated, is then used to quantify the molar amount of aldehyde and ketone, expressed as mg KOH per gram sample. To calibrate the carbonyl number instrument, typically three standards of 2-octanone (98% purity) in pure 1-octanol are used (0.1, 0.2 and 0.3 CBN). If the CBN of a solution approaches 0.3, additional samples should be diluted before testing.

This method suffers from several disadvantages. Among these are: large quantities of chemicals are needed to run the continuously operating instrument; the instrument uses chart paper and a logarithmic scale to derive the carbonyl number, which limits the practical range of the scale from 0.00 to 0.30 mg KOH/g sample; the initial cost and maintenance costs of the instrument are high, two time-consuming calibrations are required daily and the calibration curve is typically not linear; and the turnaround time for one sample is typically available no sooner than 45 minutes after the sample preparation.

Thus, the current laboratory analysis to determine the carbonyl content of Oxo alcohols is labor-intensive and time-consuming, reducing the economies of the process. What is needed is a more rapid method to determine the carbonyl content that would provide for at-line process control.

A reagent comprising an alcoholic solution of 2,4-dinitrophenylhydrazine (DNPH) and sulfuric acid has previously been described for qualitative analysis using Thin Layer Chromatography (TLC). See Organikum, p. 70-71, 16, VEB Verlag, Berlin 1986. This technique, however, is inapplicable to quantitative determination.

The present inventors have surprisingly discovered a new method for quantitative analysis of carbonyl utilizing a reagent comprising, in a preferred embodiment, an alcoholic solution of DNPH and sulfuric acid, and a procedure that may be specially adapted, in preferred embodiments, to be carried out in a few minutes. Furthermore, the technique and preferred instrumentation are easily transportable to the field, and thus the determination may be carried out at-line.

### SUMMARY OF THE INVENTION

In an embodiment, the invention is directed to an improved process for quantitative analysis of an analyte (species of interest, e.g., carbonyl) comprising mixing at least a part of a solution to be tested (the sample) with a reagent comprising a first species that will react with said analyte to form a second species that may be quantitatively determined, said reagent further comprising an acid capable of catalyzing the reaction of said analyte and said first species to form said second species, wherein said acid catalyzes said reaction faster than HCl. In a preferred embodiment the analyte is a carbonyl-containing compound. In a more preferred embodiment the process further comprises a step of reacting said second species with a strong base, such as KOH, to form a third species, and measuring the quantity of said third species using a spectrophotometric technique, even more preferably using a calorimeter, and still more preferably the quantitative determination is made using the yellowness color index according to ASTM E-313. By measurement of the quantity of said second or third species, the quantitative determination of the analyte is determined by correlation, as would be readily apparent to one of ordinary skill in the art.

In a preferred embodiment, the invention is directed to a method for determination of carbonyl content in a sample characterized by the addition of sample to a reagent comprising a first species, such as a phenylhydrazine, that will quickly form a colored entity, the second species, with aldehydes and/or ketones, and an acid that catalyzes the formation of said colored entity in the presence of aldehydes and/or ketones faster than HCl, preferably sulfuric acid. In a preferred embodiment, the said second species is treated with a strong base, such as KOH, to form a colored third species. In a preferred embodiment, the carbonyl number is determined by use of a spectrophotometer, preferably a colorimeter.

In an embodiment, which may also be an embodiment of other embodiments mentioned herein, the invention is directed to a reagent including an aqueous alcoholic solution of a first species, preferably a phenylhydrazine having electron-withdrawing substituents on the phenyl ring, e.g., 2,4-dinitrophenylhydrazine (DNPH), and a strong acid, preferably an acid such as sulfuric acid that catalyzes the reaction of carbonyl-containing species with said first species faster than HCl. The reagent is preferably a concentrated solution containing 10 vol. % or more acid and about one part by weight first species to about 5 parts by volume concentrated acid. In a further embodiment the invention is directed to the aforementioned reagent having added thereto an aliquot of sample and a strong basic solution, wherein said strong basic solution is preferably an aqueous alcoholic solution containing about 45 vol. % or more water, preferably about 45 vol. % to about 55 vol. %, and about 55 vol. % or less of denatured alcohol, preferably about 55 vol. % to about 45 vol. %, with the amount of strong base (e.g., KOH) added in the range of about 1 part base to about 15 to about 30 parts by volume water, most preferably about one part by weight KOH to about 22.5 parts by volume water.

In yet other embodiments, which may also be embodiments of other embodiments mentioned herein, the invention is directed to the mixing of analyte and reagent, and the mixing of a solution comprising a second species according to the invention and a strong basic solution, using a high speed mixer at approximately 1000 rpm, preferably for about 1 minute. In a preferred embodiment, such mixing allows for rapid application of the analytical technique according to the invention without the need for external heating in a convection oven such as provided for in the prior art.

In yet another embodiment, the embodiments set forth above are used to analyze synthetic alcohols, preferably at least one synthetic alcohol selected from C4-C15, more preferably C6-C13, still more preferably C7-C13, yet still more preferably C8-C13, branched or linear synthetic alcohols, any of the aforementioned ranges of which may be obtained, in a preferred embodiment, by the Oxo Process.

In a preferred embodiment, the samples tested have a carbonyl number in the range of 0 to 0.8.

In further embodiments the invention is directed to the use of alcohol compositions improved by the analytical technique according to the invention, especially for use in making surfactants, plasticizers, lubricants, and the like, and still further to compositions and articles comprising said surfactants, plasticizers, and lubricants.

It is an object of this invention to provide a simple and effective process quantitative analysis, particularly adaptable to the quantitative analysis of carbonyls, and even more particularly adaptable to the finishing process of synthetic alcohols so as to improve their color and remove color-forming impurities therefrom.

Another object of the invention is to provide an analytical reagent comprising, in a preferred embodiment, DNPH and sulfuric acid, and in another preferred embodiment a highly concentrated solution of acid and a species that reacts with the analyte to form a second species that can be quantitatively determined by various techniques, wherein said reagent can be quickly mixed with a sample in the field to provide a solution useful in obtaining qualitative determination of an analyte in a matter of minutes.

These and other embodiments, objects, features, and advantages will become apparent as reference is made to the following drawings, detailed description, examples, and appended claims.

### BRIEF DESCRIPTION OF THE DRAWINGS

In the accompanying drawings, like reference numerals are used to denote like parts throughout the several views.

FIGS. 1A and 1B are perspective views from the top and side, respectively, of the adapter for holding samples for the colorimeter from Hunter, according to the invention.

FIGS. 2A and 2B are perspective views from the top and side, respectively, of the adapter for holding samples for the high speed vortex mixer from Fisher, according to the present invention

### DETAILED DESCRIPTION OF THE INVENTION

In an embodiment, the present invention is directed to the analysis of an sample for an analyte comprising addition of at least a portion of a solution to be tested (the sample) for the quantitative determination of said analyte to a reagent solution comprising an active species (or first species) that will form an entity (or second species) in the presence of said analyte, wherein said entity may be quantitatively determined by spectroscopic or other techniques, the improvement comprising a reagent solution which, in an embodiment comprises, and in another embodiment consisting essentially of, said active species and an acid that will catalyze the formation of said entity in the presence of said analyte faster than HCl. In an embodiment, a strong base such as KOH is then added and the carbonyl number of the thus-treated solution is determined by an appropriately calibrated spectrometer, e.g., a Hunter calorimeter, particularly by the yellowness color index using ASTM E-313. To avoid misunderstanding, as used herein the term “analyte” means the species that is being quantitatively analyzed, e.g., aldehydes and/or ketones in a sample comprising C5-C15 alcohols.

The species that will form a second entity, preferably a colored entity, with the analyte, preferably aldehydes and ketones, whereby said analyte may be quantitatively determined by quantitative determination of said second entity, either alone without further reaction (e.g. by non-extractive or extractive analysis such as set forth in Bartkiewicz et al., or Lohman, respectively, referred to above) or by reaction of said second entity with yet another compound, e.g., a strong base, preferably KOH, to generate a third entity which may subsequently be quantitatively determined by techniques such as any spectroscopic method, is preferably an active amine derivative such as a phenylhydrazine, preferably a phenylhydrazine having electron-withdrawing substituents, e.g., a nitro group, such as a dinitrophenylhydrazine, most preferably 2,4-dinitrophenylhydrazine. This species that will form a third and preferably colored entity may also be referred to herein as “the color-forming entity”. As used herein “DNPH” refers specifically to the species 2,4-dinitrophenylhydrazine. In an embodiment, the species that will form a colored entity with aldehydes and ketones will be a species that, after forming a colored entity with any aldehyde and/or ketone present in an aliquot of the solution sampled, will react with KOH to form a species that may be analyzed by spectrophotometric techniques, preferably using the yellowness color index measure according to ASTM E-313. The yellowness color index is per se well-known; see, for instance, U.S. Pat. No. 3,972,854.

It will be recognized by one of skill in the art in possession of the present disclosure that a color-forming entity may be caused to react with an analyte comprising a moiety of interest other than an aldehyde or ketone and which may also be analyzed by a spectroscopic technique, e.g., carboxylic acid groups by IR spectroscopy, and the like. In particular, carbonyl derivatives having the formula X—C(O)—Y (where X and Y, which may be the same or different, are independently selected from H, F, Cl, Br, I, OR, SR, SeR, NRR′, PRR′, CRR′R″, SiRR′R′, BRR′, AlRR′, where R, R′, and R″, which may be the same or different, are independently selected from H, B, Al, C, Si, N, P, O, S, Se, F, Cl, Br, I) will react with the first species according to the invention, e.g., DNPH, to form derivatives that can be analyzed using extraction or non-extractive quantitative analysis by chromatographic (e.g., GC, HPLC, or Super Critical Fluid Chromatography (SFC)) and/or spectroscopic techniques (e.g., IR, UV-vis, Raman, NMR, or colorimetry). The structure X—C(O)—Y will be recognized by the ordinary artisan to mean an X and Y substituent independently bonded to the carbon atom of the carbonyl group C(O), otherwise indicated by the structure C═O.

The reagent comprising the first species, preferably a color-forming entity, will also comprise an acid. In a preferred embodiment, the acid is an acid that catalyzes the reaction between the first species and the analyte faster than HCl. A DNPH.HCl complex has been used in the prior art but the present inventors have discovered that in one embodiment of the invention a more robust reaction is necessary in order to provide for an at-line analysis on the order of minutes. Sulfuric acid is the preferred acid.

In another preferred embodiment, the reagent will be a highly concentrated solution of acid and first species. In this embodiment, the reagent comprises a solution containing 10 vol. % or more acid and about 1 part by weight first species to about 5 parts by volume concentrated acid. In a preferred embodiment, the acid is an acid that catalyzes the reaction of the analyte, if present, and said first species faster than HCl. Thus, in a preferred embodiment, the reagent comprises 10 vol. % or more sulfuric acid and about 1 g DNPH per 5 mL sulfuric acid in an aqueous alcoholic solution. Other acids useful in this embodiment include HCl, HClO₃, HNO3, HClO₄, trifluoromethylsulfonic acid, and the like.

In another preferred embodiment, which may be a preferred embodiment of either embodiments of the previous two paragraphs, a solution according to the present invention, useful for the quantitative analysis of an analyte according to the present invention, comprises the reaction product, if any, of the aforementioned solution comprising strong acid (e.g., H₂SO₄), a first species that will react with an analyte (e.g., carbonyl-containing species) to form a second species that may be quantitatively determined. The said second species may then be caused to react with a strong base (e.g., KOH) to form a third species. In the case where the sample contains a carbonyl-containing species and the first species is DNPH, the final strong base solution will comprise such a third species, which (without wishing to be bound by theory) is believed to be the “chinoidal anion” shown below.

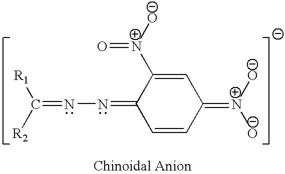

In another preferred embodiment the solvent for the reagent comprising the first entity, e.g., color-forming entity, and the acid is an aqueous alcohol solution, preferably a mixture of water and ethanol. It is preferred that the alcohol be denatured alcohol and that the water be deionized water. In an embodiment, a mixed solvent useful in the present invention is a solution having a ratio of ethanol:water of from about 4:1 to about 1:1, and in a preferred embodiment the solvent will comprise about 3 parts ethanol to about 1 part water. The preferred denatured alcohol is available from EMD as product AX0445E-1, a high purity solvent consisting of approximately 95 parts by volume of specialty denatured alcohol formula 3A (200 proof), methanol (in the amount of about 4.3 vol. % in the final high purity solvent) and 5 parts by volume isopropyl alcohol (IPA).

By way of example which is not intended to be limiting, a reagent “A” comprising a strong acid, such as sulfuric acid, and the first species, such as DNPH, in an aqueous alcohol solution is prepared. A strong base solution “B” comprising, in a preferred embodiment, 0.1 g KOH in 5 mL aqueous alcoholic solution, is also prepared. Approximately 1 mL “A” and 1 mL of a “sample”, for example an aliquot taken of C6-C13 alcohol(s) from the finishing section of an Oxo hydrogenation section, are mixed together. In the case where “sample” comprises at least one species having a carbonyl moiety, the mixing generates the “second species”. In one embodiment of the present invention, this second species may be extracted, e.g., using hexane, and subsequently analyzed quantitatively for CBN, or in another embodiment, the solution containing the second species may be treated as follows. According to a preferred embodiment of the invention, a mixture of 100 microliter (0.1 mL) of the resultant mixture of “A” and “sample” and 5-mL “B” are mixed and, again in the case where “sample” comprises at least one species having a carbonyl moiety, the mixing generates the “third species”, which in a more preferred embodiment is the chinoidal anion shown above. In a preferred embodiment the concentration of the chinoidal anion is determined, preferably by using the Hunter colorimeter and yellowness color index according to ASTM E-313, yielding the analyte concentration by correlation as would be readily apparent to one of ordinary skill in the art in possession of the present disclosure.

In yet other embodiments, the invention is directed to the mixing of sample (which may contain analyte) and reagent, and also to the mixing of this solution of sample and reagent (which will contain the second species if analyte is present in sample) with a strong base (which will contain the third species if analyte is present in the sample) using a high-speed mixer at approximately 1000 rpm, in a preferred embodiment for about 1 minute. This rapid vortex mixing allows for rapid application of the analytical technique according to the invention without the need for external heating in a convection oven such as provided for in the prior art.

It will be recognized by one of skill in the art in possession of the present disclosure that each of the aforementioned embodiments may be combined in such a way as to provide for an even faster quantitative analysis. For example, the embodiment using the more concentrated reagent solution may be combined with the embodiment using the acid that catalyzes the reaction of the first entity with the analyte, which combination may in turn be combined with the embodiment using high speed vortex mixer, which combination may in turn be combined with a strong base solution, which combination may in turn be combined with the embodiment using the colorimeter and even more preferably the yellowness color index according to the ASTM method described herein.

Thus, in an embodiment of the present invention, a reagent solution is prepared comprising the species that will form a colored entity with aldehydes and ketones, e.g., DNPH, and the acid catalyzing the reaction faster than HCl, e.g., H₂SO₄, and a solvent, e.g., an aqueous alcohol solution. It will be understood by one of ordinary skill in the art wishing to follow safe laboratory practice that a small amount of the acid stronger than HCl is slowly added to the aqueous alcohol solution. The color-forming species is typically then added and the mixture is well stirred. The amount of each of the ingredients may be determined by routine experimentation by one of ordinary skill in the art in possession of the present disclosure. This reagent solution may be prepared well ahead of the time at which the analysis will occur.

In an embodiment, an aqueous solution comprising a strong base is also prepared. Conveniently, the strong base will be KOH, but other strong bases such as NaOH and the like may be used. The basic solution will also preferably comprise alcohol, preferably the same alcohol as used in the reagent comprising the color forming entity, above, i.e., in an embodiment, denatured ethanol. A convenient preparation of an aqueous alcoholic base solution is described in detail in the experimental section below, but again, the exact ingredients and amounts used in preparing the basic solution may be determined by one of ordinary skill in the art in possession of the present disclosure. The only critical nature of the basic solution is that it cause a reaction with the species formed from the reaction of the, e.g., color-forming entity and the aldehyde and/or ketone, to form a ionic species that may be preferably analyzed by spectrophotometric techniques.

The quantitative analytical technique according to the invention is conveniently and advantageously adapted to a commercial Oxo Process finishing section. In this embodiment, the samples to be analyzed according to the process of the invention preferably are samples from a commercial Oxo Process hydrogenation section. However, it is to be understood that the process according to the invention is useful for quantitative analysis, e.g., carbonyl number determination on any sample by the addition of the reagent according to the present invention followed by potentiometric titration or spectrophotometric techniques using extractive or non-extractive methods, and is also useful for the determination of other analytes, i.e., those analytes which react with the reagent according to the present invention to form a moiety which may be quantitatively analyzed by spectroscopic (or spectrophotometric; the terms are used interchangeably herein), chromatographic, or other quantitative techniques.

In an embodiment of the invention, which may conveniently be adapted to the Oxo Process finishing section, an aliquot of the sample to be analyzed for one or more species of interest (the analyte) is collected and mixed with the reagent containing the color-forming entity, preferably a reagent comprising the color-forming entity, the acid catalyzing the relevant reaction faster than HCl, and the aqueous denatured alcohol solvent. The mixture is conveniently shaken or stirred in a capped vial. In the preferred embodiment the mixture is mixed in a high speed vortex mixer at about 1,000 rpm for about 1 minute. Typically the samples taken will be on the order of a milliliter and in a preferred embodiment aliquots are taken by using the appropriately sized Eppendorf pipettes. In a preferred embodiment the ratio of sample to reagent is from about 2:1 to about 1:2, more preferably about 1:1. Again, the exact details of this step may be ascertained by one of ordinary skill in the art in possession of the present disclosure without more than routine experimentation.

At this point, the analyte may be quantitatively determined by, for instance, a quantitative chromatographic method, such as GC, HPLC, or SFC, using various commercially available detectors, or it may be further processed, as described below, and analyzed by an extractive or non-extractive technique using a spectrophotometer, such as a colorimeter, UV-vis, Raman, NMR, or infra-red (e.g., FTIR) instrument.

In a preferred embodiment, an aliquot of the solution just prepared is then mixed with the basic solution by shaking or stirring. Again, in a preferred embodiment the mixing is accomplished by use of a high speed vortex mixer capable of mixing at the rate of about 1,000 rpm. By use of such vortex mixing, this step may be accomplished in approximately one minutes. In the procedure outlined in more detail below, used to analyze synthetic alcohols, particularly C6-C13 Oxo alcohols, the ratio of analyte solution comprising the color-forming reagent prepared in the previous step to basic solution is on the order of about 1 part to about 50 parts, but the specific ratios will depend on the details of the entire procedure and can be ascertained by one of ordinary skill in the art in possession of the present disclosure without more than routine experimentation.

Typically, in the analysis of Oxo aldehydes by the procedure according to the present invention, a black solution is formed upon mixing of the analyte solution and basic solution, followed by the formation of a stable yellow-brown solution upon continued mixing, the latter of which may then be analyzed by, with or without extraction, by a spectrophotometric technique. It is preferred that after addition of KOH the solution is mixed using a high speed mixing apparatus, preferably at 1000 rpm for one minute using a vortex mixer described herein, followed by drawing the mixture into a disposable syringe, preferably a 5-mL syringe with a Luer-Lock connection. Then a filter is attached to the syringe, preferably a 0.45 μm PTFE filter with a Luer-Lock connection, and then the solution is pushed through the filter into a container, preferably a 7-mL scintillation vial, followed by quantitative determination in, for instance, a calorimeter. Using preferred embodiments of the invention, the skilled artisan may accomplish this within 1 minute of removal from the high speed mixing apparatus.

It is preferred that transfer of solutions occur using Eppendorf pipettes, but other transfer devices would be known to those of skill in the art. Typically vials in which the various solutions above are mixed may be standard laboratory vials appropriate for the volumes used, they may be vials supplied by the spectrophotometry equipment suppliers, e.g., scintillation vials

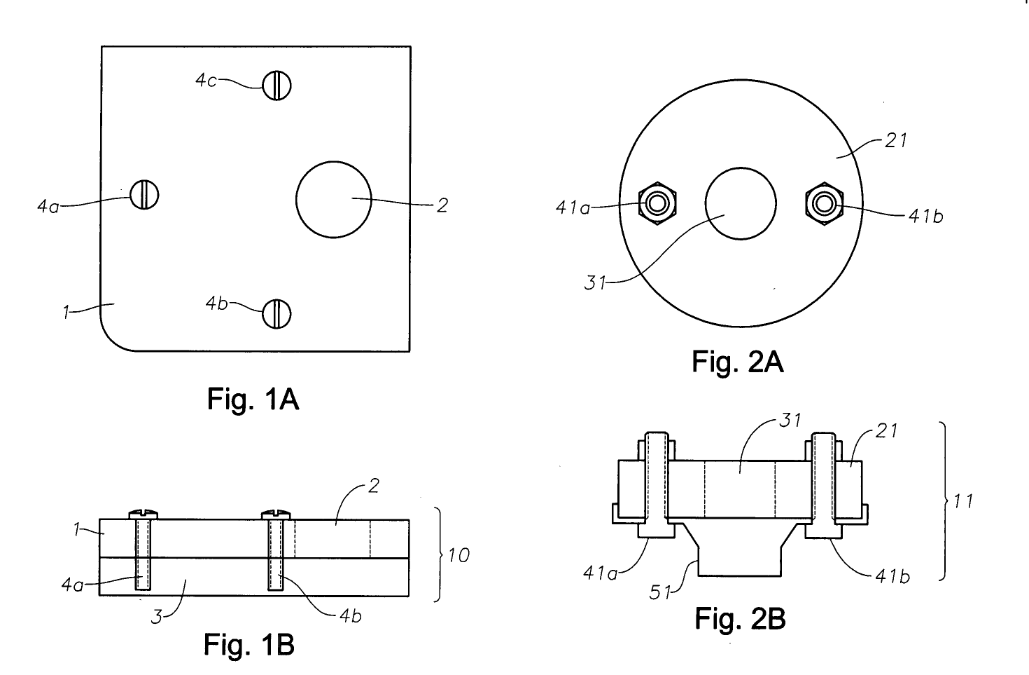

In another aspect of the present invention, the aforementioned 7-mL vial is used to minimize the sample requirements on the spectrophotometer, e.g., colorimeter. Typically the original equipment manufacturer supplies vials that require a large amount of material, relative to the amount of material used in a preferred embodiment of the present invention. Since the specially prepared vial is much smaller than those commercially manufactured to be useful in a typical colorimeter, a sample holder adapter must be prepared, as shown in FIG. 1A (top view) and FIG. 1B (side view).

In FIGS. 1A and 1B, show an adapter 10 from the top view and side view, respectively, shaped to fit into the sample holder of the calorimeter. Adapter 10 comprises at least one part 1 having an opening 2 formed in the adapter plate of sufficient diameter to hold the specially-prepared scintillation vial, e.g., in the preferred embodiment the diameter is about 16 mm to hold a 7-mL scintillation vial. The adapter 10 may be manufactured of any material, preferably Plexiglas™ or similar material which is lightweight and easily manipulated. The opening 2 may be formed, for instance, by drilling using an appropriately sized bit. In an embodiment, the part or plate 1 may be supported on a matching plate 3 which comprises the adapter 10. Plate 3, if used, may be manufactured using a more rigid but preferably lightweight material, such as aluminum. Plates 1 and 3 are attached via plural bolts 4a, 4b, 4c, etc., or the two plates may be attached by some other method such as by use of adhesives. The bottom of the sample vial may be placed in the adapter opening 2 and then the machine operated according to instructions supplied therewith.

Similar to the aforementioned adapter for the spectrophotometer, an adapter may be prepared for the mixer so that samples may be mixed, e.g., in the 20-mL scintillation vials previously described. An example of such an adapter is shown in FIGS. 2A (top view) and 2B (side view). FIG. 2A shows the top view of a modified sample holder 11 for the Fisher Scientific Digital Deluxe Mini Vortex Mixer used in preferred embodiment of the invention. The sample holder 11 comprises adapter part 21 having opening 31 of sufficient shape to hold a sample vial of choice, e.g., a 20-mL scintillation vial. The part 21 is preferably comprises of a soft foam such as polyurethane to provide a “forgiving” surface for a glass container to be vortexed. Part 21 may be attached to a connecting part 51 (shown in FIG. 2B), conveniently made of PVC and supplied by Fisher Scientific, by plural bolts 41a, 41b, etc. It will be recognized that other means of connecting 21 and 31 may be used, such as adhesives.

Mixing and/or stirring as described herein may be accomplished by the standard methods of capping a vial filled with the material to be mixed and shaking the vial, or by adding a stir bar and setting the solution on a magnetic stirrer. However, in a preferred embodiment, super efficient and high speed mixing may be accomplished using a “vortex” mixer, such as a Digital Deluxe Mini Vortex Mixer (#12-810-3 available from Fisher Scientific) capable of spinning a sample at 1000 rpm.

Experimental

The following examples are meant to illustrate the present invention. Numerous modifications and variations are possible and it is to be understood that within the scope of the appended claims, the invention may be practiced otherwise than as specifically described herein.

### COMPARATIVE EXAMPLES

Comparative examples were prepared and analyzed as set forth above in the Background section (i.e., in the section describing spectrophotometric determination using a Bran and Luebbe AA2). The exact protocol is set forth in the publication BRCP 4589, available from ExxonMobil Chemical Company.

### Examples According to the Present Invention

Examples according to the present invention were prepared and analyzed as described hereinbelow. The exact protocol is set forth in the publication BRCP 4588, available from ExxonMobil Chemical Company.

(A) The DNPH solution reagent solution was prepared. To a one quart brown glass bottle was added 600 mL of denatured alcohol (#AX0445E-1, commercially available from EM Science). To the alcohol, was slowly added 120 mL of concentrated sulfuric acid (#SX1244-13, commercially available from EM Science). To the acid alcohol solution, was added 24 g of solid 2,4-dinitrophenyl-hydrazine (DNPH, #DI149, available from Spectrum Chemicals). A stir bar was added and the thus-prepared mixture was stirred until the DNPH was dissolved. (approximately 30 min). 192 mL of distilled water was added and the solution stirred for another 30 minutes.

(B) The KOH solution was prepared. 20 g of dry KOH (#PX1480-14 commercially available from EMD) was added to 450 mL of distilled water and made up to 1 L with denatured alcohol (denatured alcohol as above).

(C) 1 mL of the sample alcohol was transferred to a 20-mL scintillation vial using an Eppendorf pipette. 1 mL of the DNPH solution prepared above in (A) was then added to the scintillation vial using an Eppendorf pipette, the vial capped, and the mixture of alcohol and DNPH was shaken for 60 seconds, using a digital mini-vortexer (Time 1 min, Speed 1000 rpm), modified with the sample holder illustrated in FIG. 2.

(D) 100 microliters of the solution prepared in (C) was added to a 20-mL scintillation vial using an Eppendorf pipette.

(E) 5 mL of the KOH solution prepared in (B) was added to the scintillation vial comprising DNPH prepared in (D), the vial capped and then shaken for 60 seconds, using a digital mini-vortexer (Time 1 min, Speed 1000 rpm) modified with the sample holder illustrated in FIG. 2, described above.

(F) The solution prepared in (E) was then filtered through a 0.45 micron filter (Fisher Scientific, Fisherbrand 25 mm Syringe Filter 0.45 um, PTFE, Non-Sterile Cat.# 09-719H) into a 7-mL scintillation vial.

(G) The 7-mL scintillation vial in (F) was placed in the Hunterlab colorimeter modified using the adapter illustrated in FIG. 1, described above, and the sample therein was scanned. The instrument provides the carbonyl number in a few seconds. For best results, the colorimeter measurement should be obtained within about 2 minutes of sample preparation as the yellow-brownish colored solution obtained in (E) degrades over time.

The Hunter colorimeter was previously calibrated using 0.0, 0.1, 0.2, 0.3, 0.4, 0.5, 0.6, 0.7, and 0.8 CBN standards using a 3.0 CBN stock solution (2-octanone in 1-octanol) diluted with 0.0 CBN octanol diluent, which was 1-octanol, 29,324-5 commercially available from Aldrich. Other carbonyl components may be used to make-up CBN standard solutions, e.g., 1-octanal, other linear aldehydes, 2-ethyl-hex-2-enal, 2-ethyl-hexanal, other branched aldehydes, 3-methyl cyclohexanone, other branched ketones, and so on. It is possible that calibration curves may vary slightly depending on the carbonyl species inside the standard and thus, depending on the accuracy desired, further routine (albeit time-consuming) experimentation may be desired. Appropriate calibration of the instrument, e.g., calorimeter, is within the skill of the ordinary artisan in possession of the present disclosure. It will be recognized by the artisan having ordinary skill that numerous alternative spectrophotometric methods can be utilized.

A comparison of the carbonyl number obtained on various identical samples by both the method of the invention (“New CBN”) and the prior art (“Current CBN”) automated method is shown in Table 1, below. Both methods were calibrated with 2-octanone in 1-octanol standards. The “Alcohol Grade” tested are commercial samples of Exxal® Alcohols. Thus, Alcohol Grade 10 is Exxal® 10 Alcohol, comprising C₁₀ alcohols, Alcohol Grade 13 is Exxal® 13 Alcohol, comprising C13 alcohols, and so forth.

TABLE 1
Current CBN versus New CBN

| Sample ID | Alcohol Grade | Current CBN | New CBN |
|---|---|---|---|
| 1 | 10 | 0.28 | 0.28 |
| 2 | 10 | 0.24 | 0.25 |
| 3 | 10 | 0.20 | 0.19 |
| 4 | 10 | 0.15 | 0.16 |
| 5 | 10 | 0.11 | 0.12 |
| 6 | 10 | 0.04 | 0.06 |
| 7 | 8 | 0.01 | 0.00 |
| 8 | 8 | 0.14 | 0.13 |
| 9 | 7 | 0.03 | 0.04 |
| 10 | 7 | 0.01 | 0.02 |
| 11 | 13 | 0.60 | 0.62 |
| 12 | 13 | 0.49 | 0.52 |
| 13 | 13 | 0.33 | 0.32 |
| 14 | 13 | 0.17 | 0.17 |
| 15 | 13 | 0.10 | 0.11 |
| 16 | 13 | 0.05 | 0.05 |
| 17 | 10 | 0.33 | 0.34 |
| 18 | 7 | 0.60 | 0.60 |

Clearly the results are similar, and yet the method according to the invention provides a method, in preferred embodiments, of obtaining quantitative results an order of magnitude faster than provided by the prior art method, as well as at lower cost.

The present invention provides for an improved analytical method for quantitative determination of carbonyl number. Although the method is not limited to the Oxo Process, as applied to the Oxo Process, it provides for an improved product by way of, inter alia, more a more uniform product quality, whether borohydride or hydrogenation is used to remove residual carbonyl-containing moieties.

Accordingly, although many variations will be apparent to one of ordinary skill in the art in possession of this disclosure, preferred embodiments include: (I) a process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, wherein said acid catalyzes said reaction faster than HCl, said process being further characterized by, in more preferred embodiments (which may be combined as would be recognized by one of ordinary skill in the art in possession of the present disclosure): (a) further comprising adding a solution comprising a strong base to react with said second species to form a third species that may be quantitatively determined, still more preferably wherein said strong base is KOH and/or wherein said solution comprising a strong base further comprises an aqueous alcohol solution (and yet still more preferably wherein said aqueous alcohol solution further comprises isopropyl alcohol and methanol); (b) further comprising quantitatively determining said analyte by a method selected from: (i) non-extractive determination by a spectrophotometric or chromatographic technique, (ii) extractive determination by a spectrophotometric or chromatographic technique, and (iii) direct and/or potentiometric titration; (c) further comprising quantitatively determining said analyte by a non-extractive determination using at least one spectrophotometric technique selected from: (i) colorimetry; (ii) IR spectroscopy; (iii) UV-vis spectroscopy; (iv) Raman spectroscopy; and (v) NMR spectroscopy; (d) wherein said first species is a phenylhydrazine, preferably a phenylhydrazine having electron withdrawing substituents on the phenyl ring, such as nitro groups, and even more preferably wherein the first species is 2,4-dinitrophenylhydrazine (DNPH); (e) wherein said sample comprises at least one alcohol selected from C4-C15 alcohols, preferably C6-C13 alcohols, more preferably C7-C13 alcohols, still more preferably C8-C13 alcohols, yet still more preferably wherein any of the aforementioned alcohol ranges are alcohols made by the Oxo Process; (f) wherein said reagent is an aqueous alcoholic solution of said first species and said acid, and in embodiments wherein the alcoholic solution comprises ethanol, preferably denature ethanol, more preferably wherein the alcoholic solution consists essentially of ethanol, methanol, and isopropyl alcohol, still more preferably a solution consisting essentially of 95 parts by volume ethanol (200 proof ethanol), 5 parts by volume isopropyl alcohol, with the final solution having 4.3 vol. % methanol; (g) wherein said acid is H₂SO₄; (h) further comprising mixing in a container said sample with a reagent comprising said acid and said first species, then adding a strong base to said container, mixing the contents of said container, and then quantitatively determining said analyte by colorimetry, more preferably wherein said mixing the contents of said container is by spinning in a vortex mixer at about 1000 rpm for 1 minute, and still more preferably wherein said vortex mixer is adapted with a polyurethane holder for said container and said container is a 20-mL scintillation vial, and in a further embodiment of (h) wherein said quantitatively determining said analyte by colorimetry comprises the steps of transferring the contents of said container to a second container, then providing said second container to a colorimeter and determining said analyte by the yellowness color index using ASTM E-313, more preferably wherein said second container is a 7-mL scintillation vial and the contents of said second container are provided to a colorimeter having an adapter for a 7-mL scintillation vial within one minute, then said analyte is quantitatively determined using the yellowness index according to ASTM E-313; (i) wherein said analyte is at least one carbonyl-containing species, including embodiments wherein said analyte is selected from any one of aldehydes, ketones, and also of mixtures of aldehydes and ketones; (j) a more specific embodiment which is a process comprising: (i) providing a reagent comprising an aqueous alcoholic solution of DNPH and sulfuric acid; (ii) providing a sample to be analyzed for carbonyl content; (iii) mixing said reagent and said sample to form a solution; (iv) determining the carbonyl content of said solution by a technique selected from direct titration techniques, extractive determination techniques, and non-extractive spectrophotometric techniques, which may also be modified by any one or more of the embodiments I (a)-(j), and also particularly by further comprising a step of adding a strong base to said solution, whereby, if said sample comprises carbonyl moieties, a chinoidal anion is formed, then quantitatively determining the carbonyl content of said solution by non-extractive spectrophotometric techniques including the correlation of chinoidal anion content with the CBN; (II) in a reagent for the quantitative determination of carbonyl-containing species in a sample, the reagent comprising a strong acid and a first species that will react with said carbonyl-containing analyte, if present, to form a second species that may be quantitatively determined, wherein said acid catalyzes the reaction of said analyte and said first species to form said second species, the improvement comprising an acid which catalyzes said reaction faster than HCl, which may be modified as a product-by-process by any one or more of the preferred embodiments described in (I) (a), (d), (e), (f), (g), and (i) of this paragraph, and/or also particularly by embodiments wherein said acid is sulfuric acid and said first species is DNPH, and/or wherein said reagent comprises comprising 10 vol. % or more of a strong acid, preferably sulfuric acid and about 1 g of a first species according to the invention, preferably DNPH, per 5 mL strong acid, more preferably a solution consisting essentially of 95 parts by volume ethanol (200 proof ethanol), 5 parts by volume isopropyl alcohol, with the final solution having 4.3 vol. % methanol in an aqueous alcoholic solution, and/or wherein said aqueous alcoholic solution comprises denatured alcohol consisting essentially of ethanol, methanol, and isopropyl alcohol; (III) a solution obtainable by, or in the alternative a solution made by, mixing the reagent as described in (I) or (II), including a reagent solution described by any relevant embodiment therein, and a sample containing at least one branched or linear alcohol selected from C4-C15 alcohols, or C6-C13 alcohols, or C7-C13 alcohols, or C8-C13 alcohols, particularly any of those ranges obtained from the Oxo Process; (IV) any of the solutions specified in (III) further mixed with a strong basic solution, with particularly preferred basic solutions set forth in (I)(a) of this paragraph and also a basic solution having the aqueous alcohol characteristics described in (I)(f); (V) a composition comprising an alcohol, preferably at least one branched or linear alcohol selected from C4-C15 alcohols, or C6-C13 alcohols, or C7-C13 alcohols, or C8-C13 alcohols, particularly any of those ranges obtained from the Oxo Process, said alcohol obtainable by, or in the alternative made by a process comprising a step of analyzing a solution including said alcohol wherein said step comprises analyzing said solution for quantitative determination of carbonyl according to any embodiment of (I) of this paragraph, and also including embodiments wherein said alcohol is obtainable by a process further comprising a step of treating a solution comprising said alcohol to decrease the content of aldehydes and/or ketones, said step selected from (i) a treatment with hydrogen gas, (ii) a treatment with a borohydride salt, (iii) a mixture thereof, followed by said step of analyzing; (VI) a composition comprising a plasticizer, said plasticizer obtainable by, or in the alternative made by, a process comprising a step of providing an alcohol composition according to any embodiment set forth in (V), and also including preferred embodiments wherein said plasticizer comprises the reaction product of said alcohol and an acid selected from substituted and phthalic acids, substituted and unsubstituted phthalic anhydrides, and mixtures thereof, especially wherein said reaction product is selected from diisononyl, diisodecyl, diisotridecyl, di-2-ethylhexyl, di-2-propylheptyl phthates and mixtures thereof, and/or wherein said alcohol is obtainable by a process comprising at least one step of treatment with a reducing agent selected from hydrogen, borohydride salts, and mixtures thereof.; (VII) a composition comprising a surfactant, said surfactant obtainable by, or in the alternative made by, a process comprising a step of providing at least one alcohol composition according to any embodiment set forth in (V) of this paragraph, and also including preferred embodiments wherein said at least one alcohol composition is selected from compositions comprising 2-propylheptanol, isononanol, isodecanaol, 2-ethylhexanol, isotridecanol, and mixtures thereof, and/or wherein said surfactant comprises the reaction product of said alcohol and at least one species selected from ethylene oxide and oligomers and polymers of ethylene oxide, and mixtures thereof, and/or wherein said alcohol is made by a process comprising at least one step of treatment with a reducing agent selected from hydrogen, borohyride salts, and mixtures thereof; (VIII) in a process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, the improvement comprising an acid that catalyzes said reaction faster than HCl, and also improvements as set forth in this paragraph by any relevant embodiments set forth in (I); (IX) in a process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, the improvement comprising a reagent comprising 10 vol. % or more of a strong acid such as sulfuric acid, and about 1 g of a first species according to the invention, such as DNPH, per 5 mL strong acid in an aqueous alcoholic solution, with further embodiments the improvements described in this paragraph by relevant embodiments in (I), but especially an embodiment wherein said aqueous alcoholic solution comprises denatured alcohol consisting essentially of ethanol, methanol, and isopropyl alcohol, and preferred aqueous alcoholic solutions as described elsewhere in this paragraph, and/or including a step of mixing said reagent with a strong base (such as KOH, LiOH, NaOH, and the like) whereby said second species, if present, forms a third species that can be quantitatively determined; and (X) in a process for quantitative determination of an analyte according to the present invention further including a step of correlation of the quantity of said analyte by reference to a calibration curve prepared by running standard solutions, wherein the standard solutions comprise linear aldehydes, branched aldehydes, linear ketones, branched ketones, and mixtures thereof, in zero carbonyl alcohols, more particularly wherein said standard solutions include at least one species selected from octanal, 2-ethyl-hex-2-enal, 2-ethyl-hexanal, nonanal, 2-propylheptanal, 3-methyl cyclohexanone, 2-octanone, and mixtures thereof, and/or wherein said zero carbonyl alcohols include at least one species selected from 1-octanol, 2-ethyl-hexanol, 2-propyl-heptanol, and mixtures thereof. Also considered preferred embodiments of the invention are lubricant compositions making use of C4-C15 synthetic alcohols as set forth in this paragraph. For the avoidance of misunderstanding, the terms “strong acid” and “strong base” means that the species exist in essentially 100% ionic form in water.

Trade names used herein are indicated by a ™ symbol or ® symbol, indicating that the names may be protected by certain trademark rights, e.g., they may be registered trademarks in various jurisdictions.

All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

The invention has been described above with reference to numerous embodiments and specific examples. Many variations will suggest themselves to those skilled in this art in light of the above detailed description. All such obvious variations are within the full intended scope of the appended claims.

We claim:

1. A process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, wherein said acid catalyzes said reaction faster than HCl.

2. The process of claim 1, further comprising adding a solution comprising a strong base to react with said second species to form a third species that may be quantitatively determined.

3. The process of claim 2, wherein said strong base is KOH.

4. The process of claim 2, wherein said solution comprising a strong base further comprises an aqueous alcohol solution.

5. The process of claim 4, wherein said aqueous alcohol solution further comprises isopropyl alcohol and methanol.

6. The process of claim 1, further comprising quantitatively determining said analyte by a method selected from: (a) non-extractive determination by a spectrophotometric technique, (b) extractive determination by a spectrophotometric technique, (c) direct and/or potentiometric titration.

7. The process of claim 1, further comprising quantitatively determining said analyte by a non-extractive determination using at least one spectrophotometric technique selected from: (a) colorimetry; (b) IR spectroscopy; (c) UV-vis spectroscopy; (d) Raman spectroscopy; and (e) NMR spectroscopy.

8. The process of claim 1, wherein said first species is a phenylhydrazine.

9. The process of claim 1, wherein said first species is DNPH.

10. The process of claim 1, wherein said sample comprises at least one alcohol selected from C4-C15 alcohols.

11. The process of claim 1, wherein said reagent is an aqueous alcoholic solution of said first species and said acid.

12. The process of claim 1, wherein said acid is H₂SO₄.

13. The process of claim 12, wherein said first species is DNPH.

14. The process of claim 1, comprising mixing in a container said sample with a reagent comprising said acid and said first species, then adding a strong base to said container, mixing the contents of said container, and then quantitatively determining said analyte by colorimetry.

15. The process of claim 14, wherein said mixing the contents of said container is by spinning in a vortex mixer at about 1000 rpm for 1 minute.

16. The process of claim 15, wherein said vortex mixer is adapted with a polyurethane holder for said container and said container is a 20-mL scintillation vial.

17. The process of claim 14, wherein said quantitatively determining said analyte by colorimetry comprises the steps of transferring the contents of said container to a second container, then providing said second container to a colorimeter and determining said analyte by the yellowness color index using ASTM E-313.

18. The process of claim 17, wherein said second container is a 7-mL scintillation vial and the contents of said second container are provided to a colorimeter having an adapter for a 7-mL scintillation vial within one minute, then said analyte is quantitatively determined using the yellowness index according to ASTM E-313.

19. The process of claim 1, wherein said analyte is at least one carbonyl-containing species.

20. The process of claim 1, wherein said analyte is selected from aldehydes, ketones, and mixtures thereof.

21. The process of claim 1, comprising:
(i) providing a reagent comprising an aqueous alcoholic solution of DNPH and sulfuric acid;
(ii) providing a sample to be analyzed for carbonyl content;
(iii) mixing said reagent and said sample to form a solution;
(iv) determining the carbonyl content of said solution by a technique selected from direct titration techniques, extractive determination techniques, and non-extractive spectrophotometric techniques.

22. The process of claim 21, further comprising a step of adding a strong base to said solution, whereby, if said sample comprises carbonyl moieties, a chinoidal anion is formed, then quantitatively determining the carbonyl content of said solution by non-extractive spectrophotometric techniques including the correlation of chinoidal anion content with the CBN.

23. In a reagent for the quantitative determination of carbonyl-containing species in a sample, the reagent comprising a strong acid and a first species that will react with said carbonyl-containing analyte, if present, to form a second species that may be quantitatively determined, wherein said acid catalyzes the reaction of said analyte and said first species to form said second species, the improvement comprising an acid which catalyzes said reaction faster than HCl.

24. The reagent according to claim 23, wherein said acid is sulfuric acid and said first species is DNPH.

25. The reagent according to claim 23, comprising 10 vol. % or more sulfuric acid and about 1 g DNPH per 5 mL sulfuric acid in an aqueous alcoholic solution.

26. The reagent according to claim 25, wherein said aqueous alcoholic solution comprises denatured alcohol consisting essentially of ethanol, methanol, and isopropyl alcohol.

27. A solution obtainable by mixing the reagent according to claim 23 and a sample containing at least one branched or linear C4-C15 alcohol.

28. The solution according to claim 27, wherein said at least one branched or linear C4-C15 alcohol is obtained from the Oxo Process.

29. A solution obtainable by mixing the solution according to claim 27 with a strong base.

30. A solution according to claim 29, wherein said at least one branched or linear C4-C15 alcohol is obtained from the Oxo Process.

31. A composition comprising an alcohol, said alcohol obtainable by a process comprising a step of analyzing a solution including said alcohol wherein said step comprises analyzing said solution for quantitative determination of carbonyl according to claim 1.

32. The composition according to claim 31, wherein said alcohol is obtainable by a process further comprising a step of treating a solution comprising said alcohol to decrease the content of aldehydes and/or ketones, said step selected from (i) a treatment with hydrogen gas, (ii) a treatment with a borohydride salt, (iii) a mixture thereof, followed by said step of analyzing.

33. A composition comprising a plasticizer, said plasticizer obtainable by a process comprising a step of providing an alcohol composition according to claim 31.

34. The composition according to claim 33, said plasticizer comprising the reaction product of said alcohol and an acid selected from substituted and phthalic acids, substituted and unsubstituted phthalic anhydrides, and mixtures thereof.

35. The composition according to claim 34, wherein said reaction product is selected from diisononyl, diisodecyl, diisotridecyl, di-2-ethylhexyl, di-2-propylheptyl phthates and mixtures thereof.

36. The composition according to claim 33, wherein said alcohol is obtainable by a process comprising at least one step of treatment with a reducing agent selected from hydrogen, borohydride salts, and mixtures thereof.

37. A composition comprising a surfactant, said surfactant obtainable by a process comprising a step of providing at least one alcohol composition according to claim 31.

38. The composition according to claim 37, wherein said at least one alcohol composition is selected from compositions comprising 2-propylheptanol, isononanol, isodecanaol, 2-ethylhexanol, isotridecanol, and mixtures thereof.

39. A composition comprising a surfactant, said surfactant obtainable by a process comprising a step of providing an alcohol composition according to claim 31.

40. The composition according to claim 32, said surfactant comprises the reaction product of said alcohol and at least one species selected from ethylene oxide and oligomers and polymers of ethylene oxide, and mixtures thereof.

41. The composition according to claim 39, wherein said alcohol composition comprises alcohols selected from isononanol, isodecanol, 2-ethylhexanol, isotridecanol, 2-propylheptanol, and mixtures thereof.

42. The composition according to claim 39, wherein said alcohol is made by a process comprising at least one step of treatment with a reducing agent selected from hydrogen, borohyride salts, and mixtures thereof.

43. In a process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, the improvement comprising an acid that catalyzes said reaction faster than HCl.

44. In a process for quantitative determination of an analyte in a sample comprising mixing said sample with a reagent comprising a first species that will react with said analyte, if present, to form a second species that may be quantitatively determined, said reagent further comprising an acid capable of catalyzing the reaction of said analyte and said first species to form said second species, the improvement comprising a reagent comprising 10 vol. % or more of a strong acid and about 1 g of said first species per 5 mL of said strong acid in an aqueous alcoholic solution.

45. The process according to claim 44, wherein said aqueous alcoholic solution comprises denatured alcohol consisting essentially of ethanol, methanol, and isopropyl alcohol.

46. The process according to claim 44, wherein said strong acid is sulfuric acid and said first species is DNPH.

47. The process according to claim 44, further comprising a step of mixing said reagent with a strong base, whereby said second species, if present, forms a third species that can be quantitatively determined.

48. The process according to claim 44, further comprising a step of correlation of the quantity of analyte by reference to a calibration curve prepared by running standard solutions comprising linear aldehydes, branched aldehydes, linear ketones, branched ketones, and mixture thereof, in zero carbonyl alcohols.

49. The process according to claim 48, wherein said standard solutions include at least one species selected from octanal, 2-ethyl-hex-2-enal, 2-ethyl-hexanal, nonanal, 2-propylheptanal, 3-methyl cyclohexanone, 2-octanone, and mixtures thereof.

50. The process according to claim 48, wherein said zero carbonyl alcohols include at least one species selected from 1-octanol, 2-ethyl-hexanol, 2-proyl-heptanol, and mixtures thereof.
